(19) United States
(12) Patent Application Publication
(10) Pub. No.: US 2011/0008303 A1
(43) Pub. Date: Jan. 13, 2011

(54) METHODS FOR TREATMENT AND PREVENTION OF MRSA/MSSA

(75) Inventor: George Y. Liu, Los Angeles, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/921,910

(22) PCT Filed: Mar. 12, 2009
(86) PCT No.: PCT/US09/36987

§ 371 Date: Sep. 10, 2010

Related U.S. Application Data
(60) Provisional application No. 61/037,187, filed on Mar. 17, 2008.

(51) Int. Cl. A61K 35/74; A61P 31/04

(52) U.S. Cl. 424/93.42; 424/93.4; 424/93.43; 424/93.45

(57) ABSTRACT

Described herein are methods for treating and preventing MRSA/MSSA. The present subject matter deviates from current treatment methods by introducing a controlled decolonization/recolonization method, which eradicates the host surface area of MRSA/MSSA, and colonizes the newly cleared surface area with a nonpathogenic or pathogenic bacteria capable of out-competing MRSA/MSSA.

### FIELD OF THE SUBJECT MATTER

The present field of the subject matter relates to methods for treatment and prevention of MRSA/MSSA. Specifically, the present subject matter relates to methods for

### BACKGROUND OF THE SUBJECT MATTER

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

MRSA is an acronym of methicillin-resistant staphylococcus aureus which is known to produce a variety of toxins and enzymes such as enterotoxin, coagulase and so forth. MSSA stands for methicillin-sensitive staphylococcus aureus and refers to all of the antibiotic sensitive strains of staph aureus, a common bacteria that can cause a wide variety of infections both in hospital and community environments. The coagulase-positive species staphylococcus aureus is well documented as a human opportunistic pathogen (Murray et al. Eds, 1999, Manual of Clinical Microbiology, 7th Ed., ASM Press, Washington, D.C.). Infections caused by staphylococcus aureus are a major cause of morbidity and mortality, especially in hospitals, nursing homes and other care facility settings. Some of the most common infections caused by staphylococcus aureus involve the skin, and they include furuncles or boils, cellulitis, impetigo, and postoperative wound infections at various sites. Some of the more serious infections produced by staphylococcus aureus are bacteremia, pneumonia, osteomyelitis, acute endocarditis, myocarditis, pericarditis, cerebritis, meningitis, scalded skin syndrome, and various abcesses.

MRSA emerged in the 1980s as a major clinical and epidemiologic problem in hospitals (Oliveira et al., 2002, Lancet Infect. Dis. 2:180-9) and continues to plague hospital settings and nursing homes. MRSA invades hospital and nursing homes through the MRSA carriers and MRSA infected patients, or through the use of normal bacterium as it is denatured into MRSA by administration of antibiotics. Onset of MRSA diseases is considered to be ascribed to direct or indirect infection among patients or from the patient to medical workers and vise versa. In particular. MRSA is transmitted through the fingers of patients and medical workers, and tools and medical devices used in hospitals and care facilities. Accordingly, a variety of chemicals have been used for disinfection of patients and medical workers, and for sterilization of various medical devices and facilities used in the hospital in order to suppress transmission of the bacteria as much as possible. However, MRSA infection continues to spread amongst patients and medical workers, identifying the need for effective reduction, or preferably elimination, of dissemination and treatment of infected patients.

Moreover, since MRSA is highly resistant to many antibacterial agents and its infection usually follows a refractory course, it further presents a serious clinical problem. Therapeutic drugs which can be prescribed for MRSA infections consist of a short list including, vancomycin, minomycin, fosfomycin, cefamethase and cefuzonam, all strong antibiotics typically used as the last line of defense. In addition, it is very likely that even these drugs will soon encounter the resistance problems experienced by common antibiotics, thus rendering the current anti-bacterialstrategies for intervention and treatment of MRSA ephemeral.

Hence, the development of a new alternative method for treatment of and proliferation against MRSA is highly desirable and required.

### BRIEF DESCRIPTION OF THE FIGURES

Exemplary embodiments are illustrated in referenced figures. It is intended that embodiments and figures disclosed herein are to be considered illustrative rather than restrictive.

FIG. 1 is a graph depicting the efficacy of precolonization against subsequent MRSA colonization in mice, in accordance with an embodiment of the present subject matter.

FIG. 2 is a timeline image depicting an experimental protocol utilized in accordance with an embodiment of the present subject matter.

### DETAILED DESCRIPTION OF THE SUBJECT MATTER

All references cited herein are incorporated by reference in their entirety as though fully set forth. Unless defined otherwise, technical and scientific terms used herein have the same meaning as commonly understood by one of ordinary skill in the art to which the subject matter belongs. Singleton et al., Dictionary of Microbiology and Molecular Biology 3rd ed., J. Wiley & Sons (New York, N.Y. 2002); March, Advanced Organic Chemistry Reactions, Mechanisms and Structure 4th ed, J. Wiley & Sons (New York, N.Y. 1992); and Sambrook and Russell, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present subject matter. Indeed, the present subject matter is in no way limited to the methods and materials described. For purposes of the present subject matter, the following terms are defined below.

“Host” may include, but is in no way limited to, any organism which may harbor, disseminate, transmit or contain MRSA/MSSA.

“Host Surface” may include, but is in no way limited to, regions of a Host infected by MRSA/MSSA. The disclosure herein has identified the nares as a possible Host Surface for illustration purposes only. Additional Host Surfaces may include, but are in no way limited to, the respiratory tract, including the nose, throat, and oral pharynx, opened wounds, insertion points of intravenous catheters, skin, nails, mucous membranes, eyes, ears, and the urinary tract.

“Outcompete” as used herein refers to the ability of a bacterium to compete for the colonization of a Host Surface and prevent secondary bacteria from colonizing the same Host Surface.

“Treatment” and “treating” as used herein refer to both therapeutic treatment and prophylactic or preventative measures, wherein the object is to eliminate and/or reduce the rate of MRSA/MSSA colonization, transmission and infection. Those in need of treatment include those already with the disorder as well as those prone to contract the infection or those in whom the infection is to be prevented.

Disclosed herein are methods that have been developed for the treatment of MRSA, and reduction of the rate of MRSA colonization, transmission and infection. The present field of the subject matter achieves this end by employing methods of bacterial interference designed to outcompete MRSA for colonization of the host surface.

MRSA is most commonly known to colonize the anterior nares of a host, although the respiratory tract, opened wounds, puncture sites, insertion points of intravenous catheters, and urinary tract are also potential sites for infection. Healthy individuals may carry MRSA asymptomatically for periods ranging from a few weeks to many years, however patients with compromised immune systems arc at a significantly greater risk of symptomatic secondary infection.

Current methods for treatment of MRSA primarily focus on the use of strong and costly antibiotics such as vancomycin to treat the resistant germs. However, the treatment of MRSA by antibiotics has significant shortcomings in that more and more antibiotics are encountering resistance issues, and MRSA may return to the host surface after antibiotic treatment is terminated,

Secondary methods for MRSA treatment employ the use of antibiotics, such as mupirocin, applied to the host surface., leading to the decolonizing of MRSA. However, the effective decolonization of MRSA by mupirocin is limited to a short term, if not repeated, which again allows for the recolonization of MRSA in the host surface. In addition, prolonged reapplication of antibiotics is impractical and inconvenient. Moreover, the effectiveness of reapplication of antibiotics may be limited by development of resistance.

The subject matter disclosed herein eliminates MRSA from the host surface by decolonization of MRSA using conventional means, such as but not limited to, the use of topical antibiotics applied to the infected host surface, eliminating MRSA, followed by the colonization of the newly cleansed host surface by a pathogenic or nonpathogenic resident bacteria which is able to outcompete MRSA. This method of treatment for MRSA is advantageous in that it may eliminate or at the least substantially reduce the population of MRSA from the host surface for a prolonged period of time, may not induce resistance (since nonpathogens known to coexist with staphylococcus aureus) and may he reapplied if necessary. Moreover, this method of treatment may reduce MRSA colonization and infection rate, reduce transmission between now decolonized hosts, and if applied at a large scale may decrease rate of colonization and infection, all of which could scale back the MRSA pandemic worldwide and significantly decrease morbidity and mortality attributed to MRSA.

In addition, the subject matter disclosed herein may lead to the use of nonpathogens to block any pathogens from colonizing the upper airway thus reducing or eliminating the incidence of ear infection, sinusitis, oral infections, and pneumonia.

In one embodiment, the MRSA infected host surface is decolonized by an antibiotic, followed by being colonized by staphylococcus epidermidis or other coagulase negative staphylococcus, such as, but not limited to, hominis, Schleiferi, Hemolyticus, Epidermidis, Capitas, Saprophyticus, Xylosus, Warneri, Schleiferi, Simulans, Sciori, Lentus, Intermedius, Cohnii and Dentocariosa. The coagulase negative staphylococcus colonized host surface may obstruct subsequent colonization by MRSA, thus eliminating or at the least substantially reducing the colonization, transmission and infection of MRSA.

In another embodiment of the subject matter, the MRSA infected host surface is decolonized by an antibiotic, followed by being colonized by corynebacterium or other antibacteria, such as but not limited to, micrococcus.

In yet another embodiment of the subject matter, the MRSA infected host surface is decolonized by an antibiotic, followed by being colonized by lactobacilli.

In another embodiment the present subject matter discloses a method for treatment of various pathogens, including streptococcus pneumoniae, haemophilus influenzae, which are known to cause upper respiratory infections.

In further embodiments the present subject matter discloses a method for treatment of allergic sinusitis by host surface decolonization with antibiotics, such as mupirocin, followed by colonization of the newly cleansed host surface by a pathogenic or nonpathogenic resident bacteria which is able to outcompete anaerobic and/or aerobic bacteria, including Staphylococcus aureus and coagulase-negative Staphylococci.

Further embodiments of the subject matter consist of the use of an antibiotic to decolonize the MRSA infected host surface, followed by being colonized by any bacterium capable of outcompeting MRSA for the host surface, including, but in no way limited to: coagulase negative staphylococcus, such as, epidermidis, hominis, Schleiferi, Hemolyticus, Epidermidis, Capitas, Saprophyticus, Xylosus, Warneri, Schleiferi, Simulans, Sciuri, Lentus, Intermedius, Cohnii and Dentocariosa; corynebacterium, such as but not limited to, micrococcus; and lactobacilli.

The present invention is also directed to a kit for the treatment of MRSA, including, but in no way limited to, (1) in subjects infected with MRSA and/or MSSA, (2) in subjects susceptible to the risk of infection of MRSA and/or MSSA and/or (3) as a preventive measure taken against colonization, transmission and/or infection of MRSA and/or MSSA. The kit is useful for practicing the inventive method of treating such conditions. The kit is an assemblage of materials or components, including at least one of the inventive compositions. Thus, in some embodiments the kit contains a composition including a nonpathogenic resident bacteria and/or a composition capable of decolonizing MRSA/MSSA, as described above.

The exact nature of the components configured in the inventive kit depends on its intended purpose. For example, some embodiments are configured for the purpose of treating the aforementioned conditions in a subject in need of such treatment. The kit may be configured particularly for the purpose of preventing treatment in subjects. In another embodiment, the kit is configured particularly for the purpose of treating infected subjects. In further embodiments, the kit may be configured for veterinary applications, for use in treating subjects such as, but not limited to farm animals, domestic animals, and laboratory animals.

Instructions for use may be included in the kit. “instructions for use” typically include a tangible expression describing the technique to be employed in using the components of the kit to effect a desired outcome, such as to treat MRSA/MSSA., including, but in no way limited to, (1) in subjects infected with MRSA and/or MSSA, (2) in subjects susceptible to the risk of infection of MRSA and/or MSSA and/or (3) as a preventive measure taken against colonization, transmission and/or infection of MRSA and/or MSSA. Optionally, the kit also contains other useful components such as diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner or the general public stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in treatment of bacterial infections. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing a nonpathogenic resident bacteria. The packaging material generally has an external label which indicates the contents and/or purpose of the kit and/or its components.

The above disclosure generally describes the present subject matter. A more complete understanding can be obtained by reference to the following Examples, which are provided for purposes of illustration only and are not intended to limit the scope of the subject matter.

### Examples

The following examples describe a range of applications of the methods of the present subject matter, as well as a number of components that may be readily integrated and/or otherwise used in connection with the same. These Examples demonstrate some of the many steps of the methods of the subject matter, and the potential impact it may have on biological studies and the conventional practice of medicine. Modifications of these Examples will be readily apparent to those skilled in the art.

### Example 1

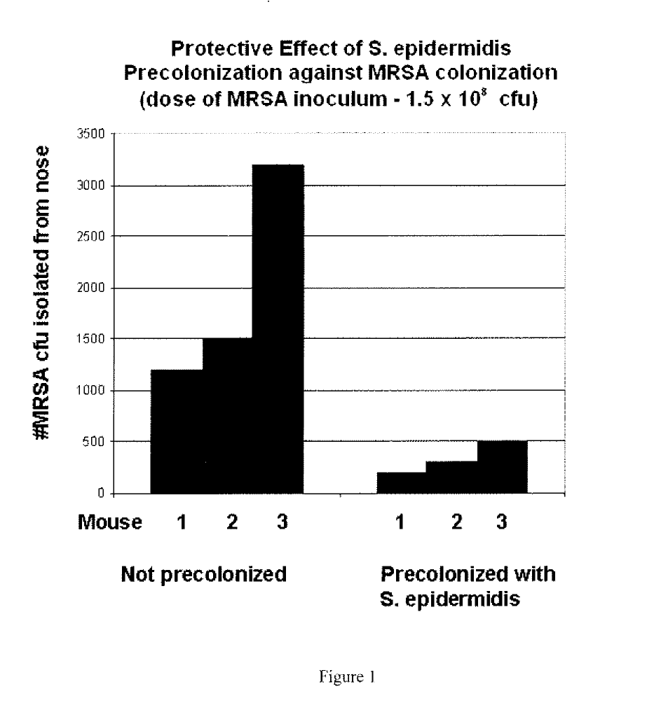

This experiment provides results of the protective effect of precolonization against subsequent MRSA colonization in mice (FIG. 1). The mice are treated with antibiotic to eradicate existing flora, including MRSA. S. epidermidis is introduced to the nares of the mice. Shortly thereafter, 1.5×10⁸ cfu dose of MRSA in introduced to the nares of the mice. The mice are sacrificed, and the incidence of MRSA in precolonized and non-precolonized nares is quantitated. The results show a very high incidence of MRSA in non-precolonized mice as compared to precolonized mice.

### Example 2

This experiment tests the effect of antibiotic treatment on endogenous nasal bacteria. To assess how effective antibiotics are at eradicating resident microbes, mice are given normal drinking water or erythromycin 50 micrograms/ml or 500 micrograms/ml. Normal or medicated water is given in the drinking water for 3 days. The antibiotic is stopped on day 4, and on day 5 mice are assessed to determine the efficacy of antibiotic treatment on the survival of resident bacteria.

### Example 3

This experiment determines the colonizing efficacy of non-pathogens. A number of non-pathogens are evaluated and include: 1) 1 strain of corynebacterium; 2) 2 strains of S. epidermidis isolated from mice; and 3) 1 strain of lactobacillus. From day 5 to day 7, after antibiotic administration, each of the non-pathogens are inoculated intranasally at a dose of 5×10⁸ cfu for three days. The mice are sacrificed two days after, and the number of surviving non-pathogens in the nares are quantitated.

### Example 4

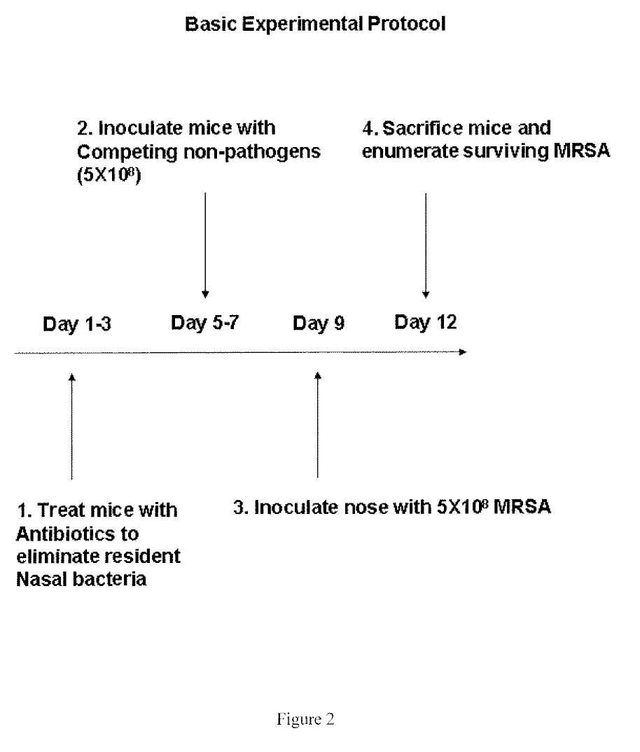

This experiment assesses the efficacy of various nonpathogens in blocking MRSA colonization. Experiment 3 is repeated except on day 9, the mice will receive 5×10⁸ cfu MRSA intranasally. On day 12, mice are sacrificed and surviving MRSA are quantitated (FIG. 2).

### Example 5

Building upon the optimization of Experiment 1-4, and identification of different strains of mice fitting for each model, different MRSA strains (HA-MRSA and CA-MRSA) are used to colonize the mouse nares, and evaluation of the blocking efficacy of various non-pathogens and pathogens are recorded and evaluated.

Various embodiments of the subject matter are described above in the Description of the Subject Matter. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms e.g., the term “including” should be interpreted as “including but not limited to,” the term “having” should be interpreted as “having at least,” the term “includes” should be interpreted as “includes but is not limited to,” etc.).

What is claimed is:

1. A method for treating a Staphylococcus aureus infection, comprising:
applying an antibiotic to a host surface containing Staphylococcus aureus; and
applying a composition comprising of bacteria to the host surface.

2. The method of claim 1, wherein the Staphylococcus aureus is selected from a group consisting of: methicillin-resistant Staphylococcus aureus, and methicillin-sensitive Staphylococcus aureus.

3. The method of claim 1, wherein the host surface comprises a mammalian epidermis.

4. The method of claim 1, wherein the antibiotic is a topical antibiotic.

5. The method of claim 1, wherein the antibiotic is mupirocin.

6. The method of claim 1, wherein the bacteria is capable of outcompeting Staphylococcus aureus.

7. The method of claim 1, wherein the bacteria is selected from a group consisting of: nonpathogenic bacteria, pathogenic bacteria, and combinations thereof.

8. The method of claim 7, wherein the nonpathogenic bacteria is selected from the group consisting of: Staphylococcus epidermidis, Staphylococcus hominis, Staphylococcus schleiferi, Staphylococcus hemolyticus, Staphylococcus epidermidis, Staphylococcus capitis, Staphylococcus saprophyticus, Staphylococcus xylosus, Staphylococcus Warneri, Staphylococcus simulans, Staphylococcus sciuri, Staphylococcus lentus, Staphylococcus intermedius, Staphylococcus cohnii and Staphylococcus dentocariosa.

9. The method of claim 7, wherein the nonpathogenic bacteria is selected from the group consisting of: actinomyces, propionibacteriurn, frankia, arthrobacter, micrococcus, lactobacilli, tnicromonospora, corynebacterium, mycobacteriutn, nocardia. rhodoeoccus, gardnerella, and streptomyces.

10. The method of claim 1, wherein the antibiotic is applied to the host surface for at least 3 days.

11. The method of claim 1 wherein the bacteria is applied to the host surface at least 48 hours after terminating application of the antibiotic.

12. A method for preventing Staphylococcus aureus infection, comprising:
applying an antibiotic to a host surface habitable to Staphylococcus aureus; and
applying a composition comprising of bacteria to the host surface.

13. The method of claim 12, wherein the Staphylococcus aureus is selected from a group consisting of: methicillin-resistant Staphylococcus aureus, and methicillin-sensitive Staphylococcus aureus.

14. The method of claim 12, wherein the host surface comprises a mammalian epidermis.

15. The method of claim 12, wherein the antibiotic is a topical antibiotic.

16. The method of claim 12, wherein the antibiotic is mupirocin.

17. The method of claim 12, wherein the bacteria is capable of outcompeting Staphylococcus aureus.

18. The method of claim 12, wherein the bacteria is selected from a group consisting of:
nonpathogenic bacteria, pathogenic bacteria, and combinations thereof.

19. The method of claim 18, wherein the nonpathogenic bacteria is selected from the group consisting of: Staphylococcus epidermidis, Staphylococcus hominis, Staphylococcus schleiferi, Staphylococcus hemolyticus, Staphylococcus epidermidis, Staphylococcus capitis, Staphylococcus saprophyticus, Staphylococcus xylosus, Staphylococcus Warneri, Staphylococcus simulans, Staphylococcus sciuri, Staphylococcus lentos, Staphylococcus intermedius, Staphylococcus cohnii and Staphylococcus dentocariosa.

20. The method of claim 18, wherein the nonpathogenic bacteria is selected from the group consisting of: actinomyces, propionibacterium, frankia, arthrobacter, micrococcus, lactobacilli, micromonospora, corynebacterium, mycobacterium, nocardia, rhodococcus, gardnerella, and streptomyces.

21. The method of claim 12, wherein the antibiotic is applied to the host surface for at least 3 days.

22. The method of claim 12, wherein the bacteria is applied to the host surface at least 48 hours after terminating application of the antibiotic.
